(19) United States
(12) Patent Application Publication
(10) Pub. No.: US 2009/0078254 A1
(43) Pub. Date: Mar. 26, 2009

(54) Anesthetic Agent Recovery

(76) Inventor: Michael Rock, Deefield, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/326,054

(22) Filed: Dec. 1, 2008

Related U.S. Application Data
(63) Continuation of application No. 10/772,757, filed on Feb. 5, 2004, now abandoned.
(60) Provisional application No. 60/537,550, filed on Jan. 20, 2004.

(51) Int. Cl. A61M 16/00

(52) U.S. Cl. 128/204.16; 128/205.27

(57) ABSTRACT

Disclosed and claimed herein are devices and method for the recovery of one or more anesthetic agents after they have been exhaled from a patient undergoing surgery and before they have been vented to the atmosphere. Typical anesthetic agents include, but are not limited to, isoflurane, desflurane, sevoflurane, and the like. Recovery of the anesthetic agents should result in numerous benefits including, but not limited to, reduction of their production costs, protection of the environment, and the like.

### CROSS REFERENCE TO RELATED APPLICATIONS

This application claims the benefit of U.S. Provisional Application No. 60/537,550, filed Jan. 20, 2004.

### FIELD

The devices and methods disclosed and claimed herein are related to the field of medicine, in particular anesthesia. Specifically, the devices and methods are related to the recovery of anesthetic agents from a waste gas stream.

### DESCRIPTION OF THE RELATED ART

As used herein anesthetic agents comprise compounds with anesthetic properties that are supplied in liquid form, but are administered to patients in need of anesthesia primarily in the gaseous state. Specifically, the methods and devices disclosed and claimed herein are for the recovery of volatile, organic, potent, inhalational anesthetic agents (VOPIAA). Such agents are typically supplied in liquid form to physicians and/or veterinarians. The agents are then poured into vaporizers on an anesthetic machine in the operating room (OR) and/or other location of use (outpatient clinic, doctor's office, oral surgery office/clinic, veterinary office/clinic, and the like). The anesthetic agents contemplated include organic compounds, as opposed to inorganic or elemental compounds (e.g., nitrous oxide and xenon). The waste anesthetic agents contemplated are not necessarily only those that are exhaled by a patient receiving the agents, in fact most of the waste anesthetic agents are never directly absorbed by a patient receiving the agents. In a typical arrangement, fresh anesthetic agent is piped in gaseous form into a breathing circuit by the anesthetic machine, where the agent mixes with the non-anesthetic gases (i.e., oxygen, etc.) in the breathing circuit. The volume of gases within the circuit is kept constant by a pressure sensitive valve. The overflow through that valve is considered the “waste gas.” This “waste gas” comprises unused anesthetic agent(s), oxygen, water vapor, etc. and is the source from which the unused anesthetic agent(s) will be recovered.

Present day volatile, organic anesthetic agents are products of advances in fluorine chemistry that attended the development of the atomic bomb. The ability to substitute fluorine for chlorine or bromine conferred greater molecular stability, lower toxicity and lower solubility (with consequent improved kinetic characteristics) to such anesthetic agents. A potential problem is that the synthetic process for producing fluorine-based anesthetic agents is both difficult and expensive.

From around 1960 to around 1980, Dr. Ross Terrel, then at Baxter, tested over 700 compounds as potential fluorine-based anesthetic agents. The 469^{th} test compound, ISOFLURANE (CHF₂—O—CHCl—CF₃), became a mainstay agent through the 1970's and 1980's and was a precursor to DESFLURANE (CHF₂—O—CHF—CF₃), the 653^{rd} test compound. Desflurane was initially produced using a potentially explosive synthesis using elemental fluorine. Another agent, SEVOFLURANE (CH₂F—O—CH(CF₃)₂), was synthesized in the late 1960's by Dr. Wallin at Travenol. Like ISOFLURANE and DESFLURANE, SEVOFLURANE is also difficult and expensive to produce.

Because of the cost of producing DESFLURANE and SEVOFLURANE, these agents were not considered viable products until the late 1980's, when outpatient surgery became a reality. Their higher fluorine content lowered their solubility and increased their onset and recovery times—a significant time improvement that more than compensated for the increased cost of production. Furthermore, these onset and recovery times could be accomplished with the same patient safety profile as ISOFLURANE.

In vitro and in vivo studies have shown DESFLURANE to be an extremely stable compound. It has two potential downfalls, namely a saturated vapor pressure near one atmosphere (1.0 atm.) at room temperature, which precludes conventional vaporizer systems, and it is about ⅕th as potent as ISOFLURANE, which means that more must be used to achieve the same effect. SEVOFLURANE has been shown to be unstable in the presence of standard CO₂ absorbents (e.g., including, but not limited to, soda lime and baralime) despite the absence of chlorination. In addition, metabolism of SEVOFLURANE (5%) produced inorganic fluoride which is potentially toxic to the renal system. Nonetheless, studies of toxicity have rarely shown any injury.

DESFLURANE and SEVOFLURANE have physical properties and physiological effects that make them ideally suited to present anesthetic practice. They are extremely safe and have rapid onset and recovery times. DESFLURANE has exceptional resistance to degradation and SEVOFLURANE is less pungent. Because of the costs of development, and the absence of a need to change present anesthetic practice, it is unlikely that any new agents will be introduced for some time. Furthermore, the difficulties and thus costs of production remain high. DESFLURANE has been off patent for three years and has not dropped in price at all.

The dangers of exposure to waste anesthetic agents that escape into the immediate environment, whether the operating room environment, the hospital or clinic environment, the physicians' office environment, the veterinary environment, and the like, have been well documented (see e.g., Criteria for a Recommended Standard, Occupational Exposure to Waste Anesthetic Gases and Vapors, DHEW (NIOSH) Publication No. 77-140 (March, 1977); Safety in the Use of Anesthetic Gases, Consensus Paper from the Basic German and French Documentation, Working Document for Occupational Safety and Health Specialists, ISSA Prevention Series No. 2042(E), (1997); Anesthetic Gases: Guidelines for Workplace Exposures, OSHA Directorate of Technical Support, The Office of Science and Technical Assessment (May, 2000); and the like).

The United States Occupational Safety and Health Administration (OSHA) mandates active extraction of all anesthetic waste gases from the operating room environment. All anesthesia machines have a scavenging apparatus that is connected to the wall suction adaptor by a hose. The waste gases are typically vented directly into the atmosphere. To date, no devices and/or methods of the type disclosed herein for extracting volatile, organic, potent, inhalational anesthetic agents from waste anesthesia gas have been disclosed.

U.S. Pat. No. 5,044,363 to Burkhart discloses an “adsorption system for scavenging anesthetic agents from waste gas released during surgical activity.” As mentioned therein, the Burkhart device consists of a cartridge loosely containing powdered activated charcoal that is connected to a conventional anesthetic administration system of the type commonly used in veterinary surgical facilities. The Burkhart device traps gases of vaporized anesthetic substances that would otherwise be released by directing those gases through the activated charcoal. The activated charcoal in the Burkhart device is loosely packed so that the container may be shaken to rearrange the activated charcoal particles to thereby generate new gas-flow paths between newly-exposed surfaces that can adsorb more anesthetic substances.

U.S. Pat. No. 6,134,914 to Eschwey et al. discloses a process and device for the “on-line recovery of xenon from anesthetic gas.” The Eschwey process and device are limited to the separation of xenon from a gas mixture. As is well known in the art, xenon is an elemental gas, significantly different than the volatile, organic gases recovered by the present devices and methods.

U.S. Pat. No. 6,364,936 to Rood et al. discloses the “selective sorption and desorption of gases with electrically heated activated carbon fiber cloth element.” The Rood device consists of a hollow enclosure containing one or more elongated hollow elements of activated carbon fiber cloth. The elements conduct electrical current and become heated to a temperature permitting selective adsorption of a gas stream constituent and subsequent desorption of the adsorbed constituent.

Anesthetic agents have been called “liquid gold” by some. Some hospitals fractionate the cost of the agent to the patient by developing a “per hour of use” charge. As mentioned before, this cost is unlikely to drop in the future because of production and tort-related expenses. Recovered agent will have a high intrinsic value and can be supplied to a manufacturer for recycling and re-refining, substantially lowering the cost of production.

In a typical Midwest city alone, there are about 55-60 hospitals with approximately 600 operating rooms (OR's) combined. There are also about 40-45 surgery centers with approximately 200 OR's combined. Cumulatively there are about 500-750 anesthesia machines in use every day. Each machine uses about ½ bottle of agent (125 cc) per day. If these numbers are extrapolated to the entire nation, and the volume of gas used, as well as gas wasted, becomes quite staggering. More than about 60,000 liters of pure anesthetic gas (100% saturated) are wasted into a typical Midwest city's atmosphere each day.

The anesthetic agent recovered by the present devices and methods can be treated as a chemical with significant potential intrinsic value. Current manufacturers may refine/re-assay the material for redistribution as a drug. This would result in a significant cost saving versus the cost of original synthesis. In addition, recovery and re-use of these expensive agents might allow for their use in less developed countries.

Thus, a need exists for methods and devices to recover, recapture, and/or reclaim volatile, potent, inhalational, organic anesthetic agents, both to protect the environment and to recover some of the cost of their production. Disclosed and claimed herein are such methods and devices.

### BRIEF SUMMARY

One aspect involves devices for the recovery of volatile, organic anesthetic agents from waste anesthesia gas. The device recovers the anesthetic agents by selectively condensing the agents in a cooling chamber and storing the condensed agents in a pressurized storage chamber. The recovered agents are available for recycling.

This aspect comprises a device for recovering one or more volatile, organic anesthetic agents from a waste anesthetic gas, the device comprising:
an entrance port for accepting the waste anesthetic gas from the anesthetic gas system;
a bypass circuit, wherein the bypass circuit is employed should the air flow in the device become blocked or the power to the device be terminated;
means for moving the waste anesthetic gas stream through the device;
a first condensation chamber for removing water vapor from the waste anesthetic gas;
means for removing the condensed water from the first condensation chamber;
a second condensation chamber for recovering the one or more volatile, organic anesthetic agents from the waste anesthetic gas stream;
means for recovering the one or more condensed, recovered anesthetic agents from the second condensation chamber;
a storage canister or storage tank for holding the recovered anesthetic agents; and
means for evacuating the remainder of the waste anesthetic gas stream from the device.

In this aspect of the device, the one or more anesthetic agent is a potent, inhalational anesthetic agent. Furthermore, the one or more anesthetic agent is selected from the group consisting of isoflurane, desflurane, and sevoflurane. Additionally, the device is connected in-line between an anesthesia machine and a vacuum port in an operating room.

The means for moving the waste anesthetic gas stream through the device is provided by one or more pumps or by a vacuum supply. Also, the means for removing the condensed water from the first condensation chamber is provided by one or more pumps and the condensed water is removed by aerosolizing/evaporating the water in a heat sink chamber. Additionally, the means for recovering the one or more condensed, recovered anesthetic agents from the second condensation chamber is provided by one or more pumps and the recovered one or more anesthetic agents are moved from the condensation chamber to a storage canister or storage tank, which is pressurizable and capable of storing 2-5 gallons of recovered anesthetic agent. The means for evacuating the remainder of the waste anesthetic gas stream from the device is provided by a wall suction port located in an operating room environment.

Another aspect involves methods for the recovery of volatile, organic anesthetic agents from waste anesthesia gas. The agents are passed through one or more condensing chambers. A first condensation chamber is cooled to a temperature such that water vapor is condensed from a gas mixture, but the anesthetic agent remains in its gaseous state. The dehumidified gas mixture comprising the anesthetic agent is routed to a second condensation at a second temperature that condenses the anesthetic agent. The recovered liquid agent is then routed to a pressurized storage chamber for subsequent recycling.

This aspect involves a method for recovering one or more volatile, organic anesthetic agents from a waste anesthetic gas, the method comprising:
collecting the waste anesthetic gas;
differentially condensing the one or more anesthetic agents from the other constituents in the waste anesthetic gas; and
recovering the one or more anesthetic agent.

Furthermore, in this aspect, the one or more anesthetic agent is a potent, inhalational anesthetic agent. Also, the one or more anesthetic agent is selected from the group consisting of isoflurane, desflurane, and sevoflurane. The condensing step is accomplished in a cooled chamber. The chamber is cooled by a process selected from the group consisting of heat exchange methods and compression/re-expansion techniques. The one or more recovered anesthetic agent is recycled and reused. Additionally, the waste anesthesia gas can be dehumidified and the dehumidification can be accomplished in a condensation chamber, wherein water is removed from the waste gas. The condensation chamber can be cooled by a process selected from the group consisting of heat exchange methods and compression/re-expansion techniques. The water can be aerosolized or evaporated into air that is heated by a hot side of a heat exchange device. Also, the one or more recovered anesthetic agents is placed into a pressurized storage canister.

### BRIEF DESCRIPTION OF THE DRAWINGS

FIG. 1 depicts an exemplary arrangement of the anesthetic agent recovery device/apparatus.

### DETAILED DESCRIPTION

### (a) Anesthetic Gas Properties

Current anesthetic practice in the United States employs the three agents previously mentioned. A significant majority of cases use DESFLURANE and SEVOFLURANE. ISOFLURANE is still used in some institutions for longer cases and for some cardiac bypass cases. HALOTHANE has mostly disappeared because of its potential to cause malignant hyperthermia, and because the byproducts of reductive metabolism (implies poor liver perfusion) can cause liver damage. It is estimated that over 90% of all cases in the United States use DESFLURANE and SEVOFLURANE. The physical properties of exemplary gases contemplated herein are tabulated below.

TABLE 1
Physical Properties of Exemplary Gases:

| Property | DESFLURANE | | SEVOFLURANE | | ISOFLURANE | |
|---|---|---|---|---|---|---|
| Formula | CHF₂—O—CHF—CF₃ | | CH₂F—O—CH(CF₃)₂ | | CHF₂—O—CHCl—CF₃ | |
| Mol. Weight | 168 | g | 200 | g | 184.5 | g |
| ml. Vapor/ml. Liquid | 641 | | 763 | | 704 | |
| MAC^{#} | 5-8% | | 1.5-2.5% | | 1-1.6% | |
| Density* | 1.465 | | 1.520 | | 1.502 | |
| Boiling Point | 22.8° | C. | 58.5° | C. | 48.5° | C. |

| | | | |
|---|---|---|---|
| SVP@18° C. | 653 | | 219 |
| SVP@20° C. | 700 | 157 | 240 |
| SVP@22° C. | 750 | | 262 |
| SVP@24° C. | 804 | | 286 |
| SVP@26° C. | 860 | | 312 |
| Odor | Pungent | “Org. Solvent” | Pungent |
| Preservative | None | Yes | None |

^{#}Minimum Alveolar Concentration of gas that prevents 50% of population from moving in response to pain. Cumulative in that adding 50% MAC N₂O will drop MAC value of agent by 50%.
*Grams per milliliter at 20° C. SVP is Saturated Vapor Pressure in mm Hg.

TABLE 2
Stability In Moist Soda Lime:

| Temperature | DESFLURANE | SEVOFLURANE | ISOFLURANE |
|---|---|---|---|
| 40° C. | Stable | Unstable | Stable |
| 60° C. | Stable | Unstable | Stable |
| 80° C. | Sl. Unstable | Unstable | Stable |

Instability in soda lime is relevant because CO₂ absorption is essential to the “closed” anesthetic circuit. There are potentially NaOH and KOH molecules from the soda lime in the circulating gases. They have the potential to degrade SEVOFLURANE over time and may need to be removed with an appropriate filter. The gas in the circuit is also close to 100% saturated with water vapor, which will also require initial removal.

In summary, each of the three typical-target anesthetic agents have different physical characteristics. Additionally, DESFLURANE boils at room temperature and so the recovered gas mixture will have to be chilled. Chilling can be accomplished using any means known in the art, including heat exchange devices as well as gas compression with re-expansion techniques.

Environmental concerns related to volatile, organic anesthetic agents are also taken into consideration. Typical volatile anesthetic agents are fluorocarbons that have a Global Warming Potential (GWP) of about 1200. On the other hand, carbon dioxide has a GWP of about 1. Given their high GWP, the U.S. Environmental Protection Agency (EPA) lists volatile anesthetic agents as medical/industrial pollutants. The language of the recommendations issued by the EPA refers only to actual levels of anesthetic agents in the operating room, physician's office/clinic, veterinary office/clinic environment. The EPA merely suggests limiting the amounts of anesthetic agent vented into the general atmosphere. The present devices and methods answer this need.

### (b) Anesthesia Machine/Operating Room Environment

Typically, the operating room (OR) is electrically insulated from the environment and power is transferred across the insulation by transformer. All electronic devices thus have to be grounded through their power cord. The anesthesia machine has limited electrical overload parameters, so only anesthesia monitoring devices are connected to the machine.

N₂O, O₂ and suction are “piped” into the OR and there are type-specific adaptors for the respective hose connectors. Typically, all three adaptors can be found in a single plate on two or more walls in the OR. The vacuum pressure is usually about −250 to about −300 mmHg. Each anesthesia machine has a vacuum hose that connects its scavenging system to the wall suction.

Exemplary anesthesia machine manufacturers include, but are not limited to, Drager Medical Inc. (Telford, Pa.; distributor of the NARKOMED series of machines), Siemens AG (Munich, Germany), and Datex-Ohmeda (Helsinki, Finland). These mobile anesthesia machines have sturdy, steel frames that could easily hold a recovery device. There are anesthesia machines that are designed to be attached to ceiling mounted swing arms; such machines could also hold a recovery device/apparatus as disclosed and claimed herein once their unique engineering challenges are addressed. While different anesthesia machines have specific scavenging system designs, they all connect by an identical nozzle to the vacuum hose. Inherent in the design is a chamber to temporarily hold gas that is exhaled faster than the vacuum can extract it. This prevents loss from the system to the OR environment. The chamber is emptied during the inhalation cycle.

Flow rates of fresh gas mixture into the anesthesia circuit vary from about 15 liters/min. to about 0.5 liters/min. The circuit is designed to vent gas at the same rate as the fresh gas is delivered into the breathing circuit. Regardless of flow, the concentration of fresh anesthetic agent will be the same as is set on the vaporizer dial. SEVOFLURANE and ISOFLURANE are delivered via normal vaporizer technology (part of the fresh gas stream is diverted through the vaporizer and picks up saturated anesthetic). DESFLURANE is heated to gaseous phase at constant temperature and pressure. It is then injected into the fresh gas stream. The amount of volatile organic anesthetic agent in the circuit depends on the rate of absorption of the agent by the patient, the fresh gas flow rate and the concentration of the agent within the fresh gas.

### (c) Exemplary Recovery System Design

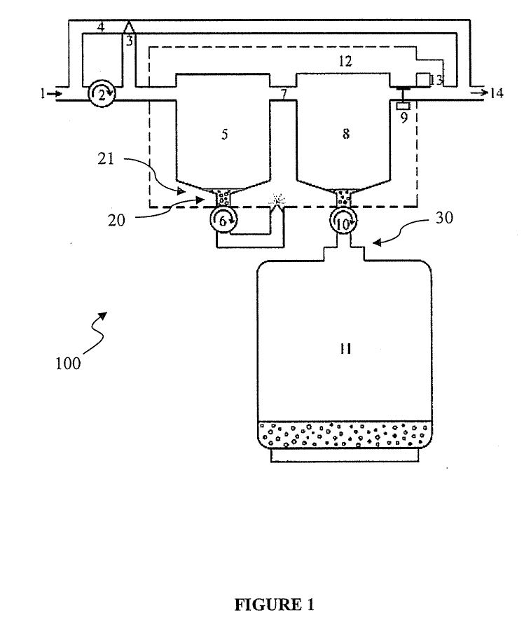

Referring to FIG. 1, the components comprising an exemplary anesthetic agent recovery apparatus/device 100 are connected as follows:

Entrance port 1 for the gaseous mixture containing one or more anesthetic agents to be recovered. The entrance port 1 is operably connected to the scavenging system port of an anesthetic machine. The scavenging system of the anesthetic machine draws away the waste gas from the patient and the fresh gas supply ensuring a constant supply of fresh gas to the patient.

Pump 2 operably connected to entrance port 1 and the remainder of the apparatus transports the gas mixture from the entrance port 1 through the remainder of the apparatus 100. The transport rate of the gas mixture through the recovery device is sufficient to remove the various gaseous flow rates from the scavenging system of the anesthesia machine, which are produced by various settings of the anesthesia machine. In addition, an ancillary system can be attached to the recovery apparatus and/or the scavenging system of the anesthesia machine to remove any brief, potentially excessive waste gas flow rates that can be caused by certain infrequent maneuvers performed by an anesthesiologist (e.g., pushing the “O₂ flush button” on the anesthesia machine and the like). Pump 2 increases pressure in the system, thereby lowering cooling requirements for condensing anesthetic agents (see e.g., the universal gas equation). The increased pressure provided by pump 2 keeps valve 3 closed and ensures flow into the condensation chambers. Should the pressure provided by pump 2 decrease significantly (e.g., by turning off power to the recovery device, etc.), valve 3 would open allowing flow through circuit 4 as a safety/bypass mechanism.

Pressure and power sensitive valve 3 operably connected to entrance port 1 and the remainder of the apparatus downstream of pump 2. If power to the apparatus is cut or if pump pressure drops, waste gas is routed from entrance port 1 through bypass circuit 4 directly to wall suction 14.

Bypass circuit 4 operably connected to entrance port 1 and wall suction 14 and used if gas flow through the apparatus is blocked or power to the apparatus is turned off.

First condensation chamber 5 operably connected downstream of pump 2 and maintained at a temperature that will condense water, but not the one or more anesthetic agents to be recovered (H₂O=less than about 100° C., DESFLURANE=22.8° C., ISOFLURANE=48.5° C. and SEVOFLURANE=58.5° C.). Cooling is provided by one or more heat exchange devices attached to coils, fins, or baffles inside the chamber 5 that increase surface area and create turbulent flow within the chamber 5. The number of coils, fins, baffles, etc. necessary for maximum dehumidification in first condensation chamber 5 can be readily determined by persons skilled in the cooling and condensation arts using any number of known techniques. A water level sensor 21 in the base of chamber 5 will activate and deactivate pump 6 as the water level rises and falls, thereby maintaining a constant level of condensate at the base of chamber 5, which will provide a barrier against the escape of gases through pump 6.

Pump 6 operably attached to a waste water port 20 of chamber 5 that intermittently aerosolizes/evaporates condensed water collected in chamber 5 into waste water vapor and evacuates the waste water vapor into heat sink chamber 12. Aerosolized/evaporated waste water vapor is evacuated from chamber 12 through port 13 to the atmosphere via wall suction 14.

Second condensation chamber 8 operably connected to first condensation chamber 5 through one-way port 7. The “dry” mixture of waste gases passes through port 7 into a second condensation chamber 8. Condensation chamber 8 is maintained at a much lower temperature than first condensation chamber 5. Subzero temperature and adequate pressure in this chamber condenses the one or more anesthetic agents to be recovered. Cooling is provided by one or more heat exchange devices attached to coils, fins, baffles, etc. inside chamber 8 that increase surface area and create turbulent flow within the chamber 8. The number of coils, fins, baffles, etc. necessary for maximum recovery of anesthetic agent(s) in second condensation chamber 8 can be readily determined by persons skilled in the cooling and condensation arts using any number of known techniques.

Pop-off valve 9 operably connected to chamber 8 that maintains a constant pressure in the device determined by the vapor pressure of the anesthetic agents, but allows “clean” gas to pass through once the agents have been removed. Valve 9 is the exit point for the gas mixture pumped into the recovery device. Valve 9 regulates pressure in the condensation chambers and thereby controls flow rates through the system. The flow rate and pressure can be coordinated by any servo-mechanism, computer-controlled mechanism, manual mechanism, etc. known to persons skilled in the relevant art. Valve 9 can be attached to a sensor in the fresh gas flow pipe that runs from the anesthesia machine outlet to the breathing circuit that is connected to the patient. The pumps, valves, heat exchangers, sensors, etc., which set the pressure(s), temperature(s), flow rate(s), etc, can be coordinated with a computer and computer software. Such computers and computer software can be readily adapted from existing computers and computer software by persons skilled in the relevant art.

Pump 10 operably connected to chamber 8 and canister 11 that intermittently empties recovered agent from chamber 8 into a storage canister 11, though one-way check valve 30.

Storage canister 11, which can withstand pressurization, operably connected to chamber 8 through pump 10 and check valve 30. A pressurizable canister of 2-5 gallons in size should be sufficient to hold enough recovered anesthetic agent for at least about 30-60 days of constant use when a typical anesthetic technique is used in humans.

Heat sink chamber 12 operably connected to the apparatus 100 to provide cooling for the hot side of the heat exchange devices in chambers 5 and 8. Cooling is provided by convective cooling from air flow derived from wall suction 14 through port 13, and by evaporative cooling from water condensed in chamber 5 and transported to heat sink chamber 12 by pump 6.

Connection to wall suction 13 operably connected to heat sink chamber 12 and wall suction 14.

Wall suction 14 operably connected to the apparatus through valve 9, port 13, and bypass circuit 4, which ranges from 250 mm Hg to about 350 mm Hg.

Any device used in the operating room will need FDA and OSHA approval—to ensure that it does not pose a threat to health. The cooling chambers 5 and 8 and other components of the device should allow unimpeded flow of gas through to wall suction 14 (even in the event of device failure), and the storage canister 11 should have a one-way valve.

Any design of the apparatus will have to contemplate numerous factors including, but not limited to, cost, ease of use, absolute reliability, ability to be adapted to known anesthesia machine configurations, and the like.

All of the references cited herein are incorporated by reference to the extent that they are not contradictory. The foregoing description of preferred embodiments of the invention is presented for purposes of illustration and explanation, and it is not intended to be exhaustive or to limit the invention to the precise form disclosed. The description was selected to best explain the principles of the invention and practical application of these principles to enable others skilled in the art to best utilize the invention in various embodiments and Various modifications as are suited to the particular use contemplated. It is intended that the scope of the invention not be limited by the specification, but defined by the claims set forth below.

What is claimed is:

1. A device for recovering one or more volatile, organic anesthetic agents from a waste anesthetic gas, the device comprising:
an entrance port for accepting the waste anesthetic gas from the anesthetic gas system;
a bypass circuit, wherein the bypass circuit is employed should the air flow in the device become blocked or the power to the device be terminated;
means for moving the waste anesthetic gas stream through the device;
a first condensation chamber for removing water vapor from the waste anesthetic gas;
means for removing the condensed water from the first condensation chamber;
a second condensation chamber for recovering the one or more volatile, organic anesthetic agents from the waste anesthetic gas stream;
means for recovering the one or more condensed, recovered anesthetic agents from the second condensation chamber;
a storage canister or storage tank for holding the recovered anesthetic agents; and
means for evacuating the remainder of the waste anesthetic gas stream from the device.

2. The device of claim 1, wherein the one or more anesthetic agent is a potent, inhalational anesthetic agent.

3. The device of claim 2, wherein the one or more anesthetic agent is selected from the group consisting of isoflurane, desflurane, and sevoflurane.

4. The device of claim 1, wherein the device is connected in-line between an anesthesia machine and a vacuum port in an operating room.

5. The device of claim 1, wherein the means for moving the waste anesthetic gas stream through the device is provided by one or more pumps.

6. The device of claim 1 wherein the means for removing the condensed water from the first condensation chamber is provided by one or more pumps and the condensed water is removed by aerosolizing the water in a heat sink chamber.

7. The device of claim 1 wherein the means for recovering the one or more condensed, recovered anesthetic agents from the second condensation chamber is provided by one or more pumps and the recovered one or more anesthetic agents are moved from the condensation chamber to a storage canister or storage tank.

8. The device of claim 1 wherein the means for evacuating the remainder of the waste anesthetic gas stream from the device is provided by a wall suction port located in an operating room environment.

9. A method for recovering one or more volatile, organic anesthetic agents from a waste anesthetic gas, the method comprising:
collecting the waste anesthetic gas;
differentially condensing the one or more anesthetic agents from the other constituents in the waste anesthetic gas;
recovering the one or more anesthetic agent.

10. The method of claim 9, wherein the one or more anesthetic agent is a potent, inhalational anesthetic agent.

11. The method of claim 10, wherein the one or more anesthetic agent is selected from the group consisting of isoflurane, desflurane, and sevoflurane.

12. The method of claim 9, wherein the condensing is accomplished in a cooled chamber.

13. The method of claim 12, wherein the chamber is cooled by a process selected from the group consisting of heat exchange methods and compression/re-expansion techniques.

14. The method of claim 9, wherein the one or more recovered anesthetic agent is recycled and reused.

15. The method of claim 9, wherein the waste anesthesia gas is dehumidified.

16. The method of claim 15, wherein the dehumidification is accomplished in a condensation chamber and wherein water is removed from the waste gas.

17. The method of claim 9, wherein the one or more recovered anesthetic agent is placed into a pressurized chamber.

18. The method of claim 16, wherein the condensation chamber is cooled by a process selected from the group consisting of heat exchange methods and compression/re-expansion techniques.

19. The method of claim 18, wherein the water is aerosolized or evaporated into to air that is heated by a hot side of a heat exchange device.

20. A device for recovering one or more volatile, organic anesthetic agents from a waste anesthetic gas, the device comprising:
an entrance port for accepting the waste anesthetic gas from the anesthetic gas system;
a bypass circuit, wherein the bypass circuit is employed should the air flow in the device become blocked or the power to the device be terminated;
one or more pumps for moving the waste anesthetic gas stream through the device;
a first condensation chamber for removing water vapor from the waste anesthetic gas;
one or more pumps for removing the condensed water from the first condensation chamber;
a second condensation chamber for recovering the one or more volatile, organic anesthetic agents from the waste anesthetic gas stream;
one or more pumps for recovering the one or more condensed, recovered anesthetic agents from the second condensation chamber;
a storage canister or storage tank for holding the recovered anesthetic agents; and
a vacuum supply for evacuating the remainder of the waste anesthetic gas stream from the device.
